(19) United States
(12) Patent Application Publication
(10) Pub. No.: US 2009/0005678 A1
(43) Pub. Date: Jan. 1, 2009

(54) Method for determining and displaying an access corridor to a target area in the brain of a patient

(76) Inventors: Kristin Schmiedehausen, Palo Alto, CA (US); Gunther Platsch, Rothenbach (DE); Thorsten Feiweier, Poxdorf (DE); Diana Martin, Herzogenaurach (DE); Sebastian Schmidt, Weisendorf (DE); Michael Szimtenings, Bonn (DE)

Correspondence Address: HARNESS, DICKEY & PIERCE, P.L.C., P.O.BOX 8910, RESTON, VA, 20195, US

(21) Appl. No.: 12/213,745

(22) Filed: Jun. 24, 2008

(30) Foreign Application Priority Data
Jun. 26, 2007 (DE) 10 2007 029 364.1

(51) Int. Cl. A61B 6/03

(52) U.S. Cl. 600/427

(57) ABSTRACT

A computer-implemented method is disclosed for determining and displaying an access corridor to a target area in the brain of a patient, as well as an imaging arrangement suited to this. In at least one embodiment, the method includes a) generating a first image of the brain via positron emission tomography, b) discriminating the target area relative to its surroundings via electronic image processing, c) generating a second image of the brain via magnetic resonance imaging while acquiring at least one anatomical structure, d) generating a third image of the brain via an imaging method displaying physiological processes for identifying at least one functional area of the brain that must not be injured in any circumstances, e) determining an access corridor to the target area while omitting the at least one functional area of the brain, and f) generating and displaying a fourth image of the brain in which the target area, the at least one functional area of the brain, the at least one anatomical structure and the access corridor are displayed, wherein steps a) to d) are carried out, one after another in quick succession, in a single frame of reference without repositioning the patient, or are even carried out simultaneously.

### PRIORITY STATEMENT

The present application hereby claims priority under 35 U.S.C. §119 on German patent application number DE 10 2007 029 364.1 filed Jun. 26, 2007, the entire contents of which is hereby incorporated herein by reference.

### FIELD

Embodiments of the present invention generally relate to a computer-implemented method for determining and displaying an access corridor to a target area in the brain of a patient, a corresponding computer program, a data storage medium on which the computer program is saved and/or an imaging arrangement for carrying out the method.

### BACKGROUND

Both neurosurgical procedures such as operations on and tissue removal from the brain, and therapeutic radiation exposure require maximum precision during the planning and when being carried out. On the one hand, a pathological finding such as a tumor or epilepsy focus is to be removed as completely as possible from the surrounding healthy brain tissue or is to be comprehensively irradiated. On the other hand, functionally important surrounding areas of the brain must be protected as well as possible. The access to an area of the brain in general describes a path from outside of the brain to the pathological finding. In practice, in one corridor there are often a plurality of accesses to the area of the brain in which the pathological finding is located and which will be referred to in the text below as target area.

One object of at least one embodiment of the present invention is to determine and display such an access corridor to the target area so that a suitable access can be selected, using medical expertise and/or aid, if required. The determination of the access corridor can be implemented only with the aid of electronic image recording and evaluation methods and systems; that is to say in general with the aid of computers.

This results in the following problems, which will be discussed successively.

First of all, the target area having the pathological finding has to be accurately delimited. Positron emission tomography—abbreviated PET—is very precise method for representing the extent and the boundaries of a brain tumor since, by way of example, biochemical changes caused by the tumor are determined. Depending on the radiopharmaceutical used, this method supplies only limited anatomical information, for example an axial location of the tumor within the brain or with reference to surrounding anatomical structures. In the case of epilepsy patients, PET is likewise an established method for identifying the focus. In this case, a change in the glucose metabolism or particular nerve actions in the respective target are used.

In order to record the necessary anatomical structures within the patient for the access corridor to the target area, magnetic resonance imaging—abbreviated MRI—can be used, as disclosed in laid-open specification DE 103 58 012 A1. Although MRI permits delimiting the tumor from the healthy tissue, it does not allow its biochemical activity to be assessed.

Furthermore, reliable identification of functionally important areas of the brain is necessary. In this case, this can relate to both functional regions of the cortex and also important nerve tracts.

Like on a map, different brain regions are assigned different functions. Usually these regions can be reliably identified on the basis of anatomical landmarks with the aid of structural imaging in the form of magnetic resonance imaging. Problems occur in the case of deviations from the norm and in particular in the case of patients whose functional areas of the brain have been displaced by a tumor, a malformation or different illnesses or results of illness and can no longer be identified unequivocally. It is even possible for certain regions, such as the speech center, to switch to the other half of the brain. With the aid of functional magnetic resonance imaging—abbreviated fMRI—it is possible to identify and anatomically assign these functionally important areas of the brain by stimulation examinations. Occasionally, in the case of foci in the patient's speech center, the patient has had to be woken up during the operation in order to reliably identify this functionally important area. If it is not possible to carry out fMRI, the course of nerve tracts and their spatial direction can be obtained by diffusion-weighted MRI or diffusion tensor imaging and suitable post-processing of the data.

All these computer-aided methods are available to the neurosurgeon or the oncologist/radiation therapist for planning and carrying out the operation or for irradiation. Since none of the mentioned techniques answer all the questions posed, the previously mentioned methods have to be carried out one after the other. A combined method is disclosed in DE 10 2005 041 381 A1. The method involves high logistical complexity and a lot of time and is burdened with a non-negligible risk of registration errors, in particular when carrying out the PET method with substances supplying few anatomical details. It is a particular disadvantage that the methods are carried out at successive times on separate systems. This means greater stress for the patient, more time and, in particular, the potential risk of inaccuracies, e.g. in the case of subsequent co-registration of the images. The patient is inevitably moved between the two recordings since different systems are used. In the previously mentioned method, the positions of the head are acquired during the recording of the positron emission data and the magnetic resonance data in spatially separated frames of reference by way of lasers.

Prior to generating a fused image, the data of the two imaging methods are respectively processed separately (reconstructed, inter alia), then registered and occasionally also subjected to geometric error correction.

### SUMMARY

At least one embodiment of the present invention improves this combined method and an imaging arrangement carrying out the method, in such a way that at least one of the previously mentioned disadvantages is avoided, in particular so that no registration of the data is required.

It is advantageous in the case of the method according to at least one embodiment of the invention that simultaneous or at least almost simultaneous isocentric acquisition of positron emission data as a first image and magnetic resonance data as a second image is carried out. By way of example, a combined MRI/PET system can be used, in which magnets define a longitudinal axis and form a part of a magnetic resonance imaging scanner, with a gradient coil and a RF-coil being arranged radially in the interior of the magnet. Gamma radiation generated by the radiopharmaceuticals is received by a multiplicity of detectors situated radially in the interior of the gradient coil and arranged along the longitudinal axis.

This positron emission data can be acquired simultaneously and/or spatially in a single frame of reference with the magnetic resonance data. Since the acquisition devices for recording magnetic resonance data and positron emission data are arranged in a single frame of reference, this additionally results in the advantage that the anatomical structures from the magnetic resonance data are automatically co-registered with the positron emission data.

In the case of simultaneous recording of the patient's brain, the magnetic resonance data and positron emission data obtained in this way can immediately be associated with each other spatially and temporally, and can thus be used later by determining and displaying an access corridor on a monitor for operation planning or irradiation planning. The term “an” access corridor should in this case to be understood to mean that it is by all means possible to also display and/or determine a plurality of access corridors. Furthermore, in order to determine a protective access corridor, a third image can still be produced beforehand by means of a method which can make physiological processes or parameters, such as perfusion changes and diffusion, visible so that functional areas of the brain can be identified. Moreover, by stimulating functional areas of the brain, for example by speaking during the recording the magnetic resonance data, their location can be determined. An access corridor to the target area omitting functional areas of the brain can thus be determined and displayed. The medical practitioner can then use this information to select a suitable access to the target area.

In addition to functional magnetic resonance records, further functionally important areas of the brain can preferably be determined or identified by means of dynamic positron emission tomography and/or functional magnetic resonance imaging (that is to say using the previously mentioned imaging apparatuses). Alternatively, the use of a further, third imaging apparatus is also possible. Regions of the brain are connected to each other by nerve tracts. If these tracts are severed during the operation or their function is disturbed, limitations of brain functions can result.

As is the case of the functional areas of the brain, nerve tracts can, in certain cases, also deviate from the norm with regard to position and orientation, by way of example in the vicinity of tumors. Information about the spatial course of nerve tracts can be obtained with the aid of diffusion weighted magnetic resonance imaging methods.

In particular, diffusion tensor imaging—abbreviated DTI—with subsequent post-processing, e.g. by fiber tracking, fiber bundle segmentation and the like, and also the BOLD (blood oxygen level dependent) method which can in particular visualize biochemical processes such as oxygen-level changes, may be mentioned here. This information aids, together with the anatomical structures, in identifying a protective access corridor to the target area determined for the operation, by means of which no functionally important nerves are injured. By means of dynamic PET, an increase or decrease in the activity of a brain region can be detected by using a suitable radioactively marked pharmaceutical (e.g. radioactively marked water or sugar). By means of magnetic resonance spectroscopy, the spatial distributions of a chemical substance and/or a ratio of two substances in the brain can be determined. Magnetic resonance imaging methods can generate data with significantly higher spatial resolution that PET methods. In the case of simultaneous recording, these areas of the brain can also be reliably associated with anatomical structures and can be taken account of when determining the access corridor.

The delimited target area and the identified area of the brain are advantageously fused in an image. By way of the simultaneous recording of magnetic resonance data and positron emission data, the mutual determination of their location with reference to anatomical structures is ensured. A protection access from the determined access corridor can be visually determined by means of the image on the monitor while taking the functional areas of the brain into account, for example. If this data is acquired in the same frame of reference, movement correction methods based on magnetic resonance imaging can additionally by used to improve the data quality of the functional PET, the weighted magnetic resonance imaging or the magnetic resonance spectroscopy.

In particular, by visualizing the delimited target area and the functional area of the brain in different colors, it is possible to ensure differentiation between the pathological target area and the functional area of the brain. The areas can be assigned to the respective imaging methods in order to distinguish between functional areas of the brain such as the speech center and nerve tracts.

The method according to at least one embodiment of the invention can be developed in such a way that the precision of the discrimination of the target area in the first image is improved by use of the second image (i.e. the magnetic resonance image). Data quality and precision which are as high as possible are essential, particularly in the brain, considering the described consequences of an operation based on false assumptions. By way of example, the positron emission data can be improved by a partial volume correction based on magnetic resonance imaging. The information obtained is used either for mutual improvement of the display or error correction. By way of example, a positron emission signal, which seems to be coming from structures such as ventricles, which are undoubtedly not regarded as signal emitters in the magnetic resonance method, can be suppressed in order to achieve a higher image quality. Errors in the magnetic resonance data due to inhomogeneities in the magnetic field can be compensated for by information from the positron emission data.

If the magnetic resonance data and the positron emission data are recorded one after the other with a short time interval between them, a common frame of reference must be provided. This can be a result of isocentric arrangement of the acquisition devices, which, for example, is ensured by a combined MRI/PET system. Likewise, the use of a stereotaxic frame is possible, so that the result data can also be used for operation planning and operation control.

An imaging arrangement according to at least one embodiment of the invention for determining and displaying an access corridor to a target area in the brain of a patient comprises a positron emission tomography imaging apparatus for generating a first image of the brain, a magnetic resonance imaging apparatus for generating a second image of the brain while recording at least one anatomical structure, an imaging apparatus imaging physiological processes for generating a third image of the brain, and a control and evaluation system for controlling the imaging arrangement as claimed in a method as mentioned above. When using the imaging arrangement according to the invention with the suitable methods, no registration of the images recorded in different ways is necessary.

### BRIEF DESCRIPTION OF THE DRAWINGS

Example embodiments of the present invention are now described in more detail with reference to the attached drawings, in which

FIG. 1 shows a schematic illustration of a first example embodiment of a method according to the invention;

FIG. 2 shows an image according to a second example embodiment of the present invention;

FIG. 3 shows, not to scale, a cross-sectional view of a brain when carrying out a third example embodiment of the method; and

FIG. 4 shows, not to scale, a cross-sectional view through an imaging arrangement according to an embodiment of the invention.

The example embodiments of the present invention will be described in the following text C with reference to the drawings.

### DETAILED DESCRIPTION OF THE EXAMPLE EMBODIMENTS

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the figures.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a, ” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Spatially relative terms, such as “beneath”, “below”, “lower”, “above”, “upper”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

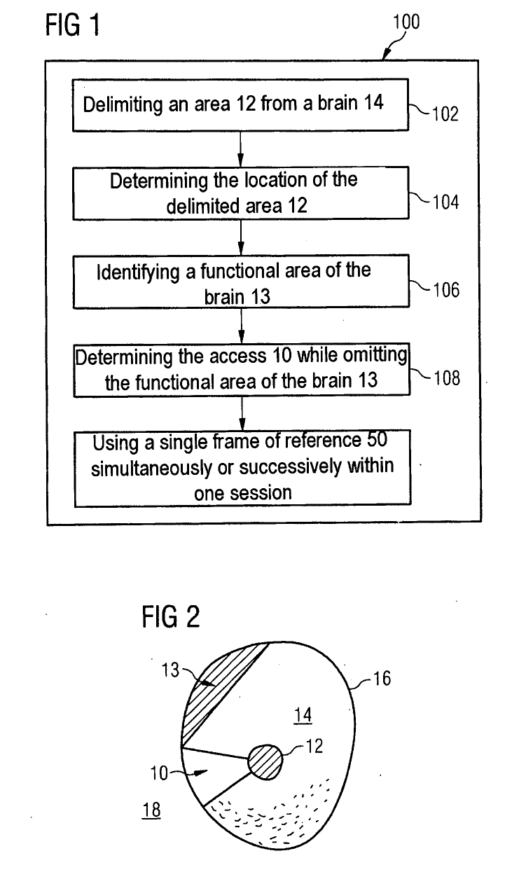

All method steps for determining an access corridor 10 to a target area 12 in a brain 14 will be explained below on the basis of FIG. 1. The method 100 according to an embodiment of the invention includes a first method step 102 for delimiting or discriminating the target area 12 by means of positron emission tomography, and, if required, additionally using MRI. In the case of PET, radioactively marked substances, which accumulate in the tumor, are injected to determine the pathological target area 12. During their radioactive decay, positrons are emitted, which recombine with electrons while emitting gamma radiation. When recording positron emission data by means of gamma ray detectors, pathologically changed target areas 12 of the brain 14 can be delimited in the blood flow. In the case of increased blood flow, a tumor is associated with the target area 12.

In order to accurately determine the location of the delimited target area 12, an image is recorded in a second method step 104 by way of magnetic resonance imaging. In particular, anatomical structures 16, such as bones, cartilage of an ear and/or an eye, can be segmented in this case, and are used for spatial assignment of the target area 12. These structures 16 contained in the magnetic resonance data are moreover important guiding structures and orientation aids for determining the access corridor 10 from outside of the brain 14. During the recording of the magnetic resonance data for determining the location, functionally important areas of the brain 13 are also included in a further method step 106 by means of so-called functional magnetic resonance imaging.

One such important area of the brain 13 is the speech center, which should be omitted during the determination of the access corridor in a subsequent method step 108, in order to be able to later select a protective access from the access corridor 10. By way of example, this is particularly important for planning a neurosurgical resection of the tumor. In order to stimulate the speech center during the recording of the magnetic resonance data, the subject is asked to say a few words, for example. Music can also be played to the subject, or the subject can perform predetermined movements of the arms and legs in order to identify other important functional areas of the brain 13. These areas of the brain 13 can be recognized as activated areas by way of magnetic resonance imaging and can be related to the anatomical structures.

The previously described method steps 102, 104, 106 can be carried out with a single so-called hybrid system. According to an embodiment of the invention, these method steps are either carried out simultaneously—that is to say in parallel with one another—or sequentially—that is to say with a short time interval between each other—in one examination cycle, that is to say without repositioning the patient. Due to this capability of simultaneous or almost simultaneous isocentric acquisition of the required positron emission data and magnetic resonance data in the same volume and with a uniform frame of reference 50, the information is thus automatically co-registered. If this information is recorded successively in a single frame of reference 50, movement correction is possible by way of the in particular temporally highly resolved magnetic resonance data.

FIG. 2 shows a particularly protective access corridor 10 to a pathologically changed target area 12 within a brain 14. In order to identify functionally important regions of the brain 13 by means of functional magnetic resonance imaging, information from a dynamic PET is furthermore included for this purpose in the method step 106. The brain 14 is supplied with a radioactively marked substance, e.g. 015-marked water, which is selectively or preferentially accumulated by the activated area of the brain 13. A further functional area of the brain represented in FIG. 2 by dots is identified by means of weighted magnetic resonance imaging such as so-called DTI and/or by means of magnetic resonance spectroscopy. In the case of diffusion weighted magnetic resonance imaging, the different mobility of water molecules in different tissue types is used.

Furthermore, the anisotropy of the mobility is used: water molecules diffuse faster parallel to nerve tracts than perpendicular to them. By suitable evaluation of the diffusion weighted data—for example, by using so-called fiber tracking—the spatial course of nerve bundles can be identified. Furthermore, the diffusion constant of water varies across different tissues. Gray brain matter and white brain matter can be distinguished in this way. Starting from the delimited target area 12, the nerve tracts in the white brain matter are identified as further functional areas of the brain 13 by means of fiber tracking. Their location is in turn determined by the anatomical structure 16 acquired by way of the magnetic resonance data.

By way of an isocentric combination of acquisition devices for recording magnetic resonance data and positron emission data, the data records of dynamic PET, DTI and magnetic resonance spectroscopy are automatically exactly co-registered. Otherwise, in the case of a sequential recording of this data, co-registration results by means of the single frame of reference 50 used in this case. In this case, the additional acquisition devices are arranged isocentrically to the detectors and coils provided in the hybrid system. This removes the risk of registration inaccuracies which can have serious consequences in the case of procedures in the brain. By way of the method according to an embodiment of the invention, an access corridor 10 for carrying out an operation or radiation therapy while omitting identified areas of the brain 13 is determined.

For improved orientation, an image 18 is generated from the previously mentioned data and information, and, in FIG. 2, provides a cross-sectional view of the brain 14. The pathological finding in the target area 12, delimited with the aid of positron emission tomography, is in this case illustrated in a different color than the identified functional areas of the brain 13. This information is displayed in a fused manner, e.g. by planning software for neurosurgical procedures and radiation therapy planning. This information is displayed out by superposition of differently colored images, which were respectively reconstructed by one of the imaging modalities.

Prior to the reconstruction of the images, delimiting the target area 12 in method step 102 and/or determining the location of the delimited target area 12 in method step 104 can be improved by means of magnetic resonance imaging and positron emission tomography respectively. Typically, a finite number of slice records are generated both in magnetic resonance imaging and in PET. The slice records have a predetermined slice thickness due to a spacing of the detectors from one another. This leads to the so-called partial volume effect, as a result of which determination of the location of the delimited target a r e a 12 does not succeed perfectly. By way of example, if the PET-slice records are recorded in the x-y direction of the single frame of reference 50, the slice thickness in the z-direction can represent different types of tissue. The determination of the location within a slice thickness in the z-direction is achieved by means of the MRI data.

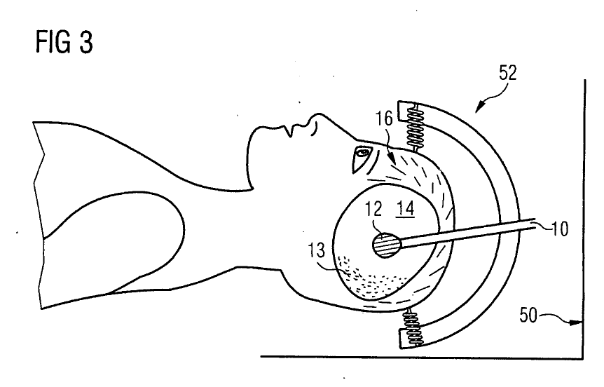

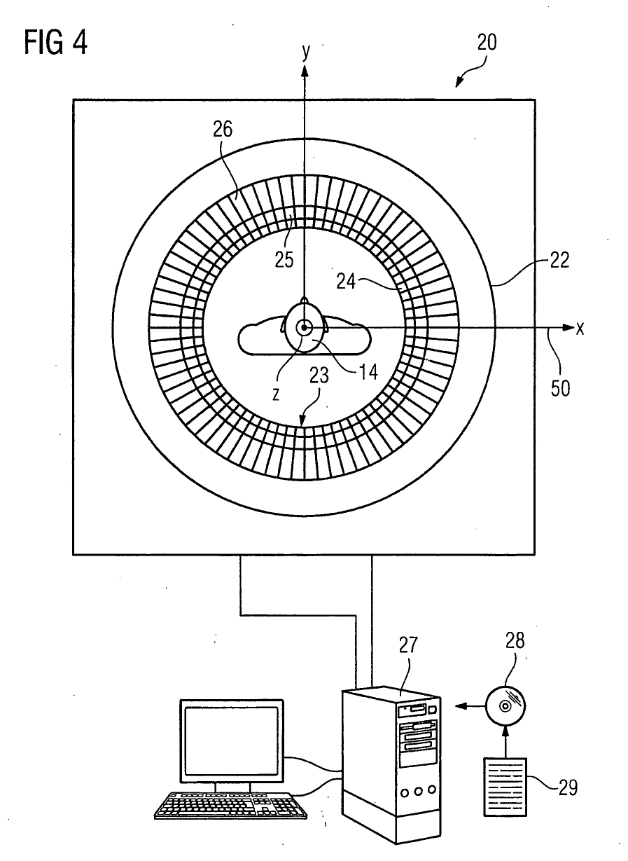

FIG. 3 shows one such frame of reference 50. According to an embodiment of the invention, this frame of reference 50 is provided by the acquisition devices of a hybrid system which allows the recording of the positron emission data and magnetic resonance data. In order to delimit an epilepsy focus in a first method step 102, the positron emission data is generated by a positron emission tomography scanner. Magnetic resonance imaging, which also acquires the frame of reference 50 of a stereotaxic frame, is used to identify anatomical structures 16. As a result of this, the target area 12 surrounding the epilepsy focus can be localized. By stimulating functional areas of the brain 13 during the recording of the magnetic resonance data, the location of these areas can likewise be determined with reference to the stereotaxic frame 52. Since these different imaging modalities are acquired using a single frame of reference 50, their relative position to one another is known, in particular in real time. The most protective access corridor 10 can be determined intraoperatively. A so-called brain pacemaker can for example also be inserted into the target area via this access corridor 10. The function can be controlled with the various previously mentioned modalities. 100481 The planning of neurosurgical procedures or carrying out radiation therapy becomes very safe and efficient with the aid of the method according to an embodiment of the invention using a combined MRI/PET imaging arrangement 20 according to FIG. 4. By combining and (at least almost) simultaneous acquisition of PET and MRI, the logistics for subsequent planning of an operation can be improved. Also, the time involved and the stress on the patient are markedly reduced. Finally, some of the previously mentioned inter-operative localization methods may no longer be required due to more precise registering and determination of the location of the important areas of the brain.

The imaging arrangement 20 according to an embodiment of the invention is a combined MRI/PET system which permits simultaneous or else only almost simultaneous and isocentric measuring of MRI data and PET data.

According to FIG. 4, the imaging arrangement 20 includes a known MRI tube 22. A plurality of PET detection units 23 are arranged mutually opposite each other in pairs along the longitudinal axis, coaxially within the MRI tube 22. Preferably, the PET detection units 23 include a photodiode array 25 with an upstream array of crystals 24 and an electrical amplifier circuit (PMT) 26. However, embodiments of the invention is not limited to PET detection units 23 having the photodiode array 25 and the upstream array of crystals 24, and differently designed photodiodes, crystals and apparatus can similarly also be used for detection.

The MRI tube 22 defines a cylindrical, first measurement field along its longitudinal direction. The multiplicity of PET detection units 23 define a cylindrical, second measurement field along the longitudinal direction z. Preferably, the second measurement field of the PET detection units 23 substantially corresponds to the first measurement field of the MRI tube 22. This is implemented for example by a corresponding adaptation of the arrangement density of the PET detection units 23 along the longitudinal axis z.

Image acquisition and processing are carried out controlled by a computer or processor 27, operated on the basis of a program 29 (symbolically illustrated as a written-on page), which is saved on a CD as a data storage medium 28, for example.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program and computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a computer readable media and is adapted to perform any one of the aforementioned methods when run on a computer device (a device including a processor). Thus, the storage medium or computer readable medium, is adapted to store information and is adapted to interact with a data processing facility or computer device to perform the method of any of the above mentioned embodiments.

The storage medium may be a built-in medium installed inside a computer device main body or a removable medium arranged so that it can be separated from the computer device main body. Examples of the built-in medium include, but are not limited to, rewriteable non-volatile memories, such as ROMs and flash memories, and hard disks. Examples of the removable medium include, but are not limited to, optical storage media such as CD-ROMs and DVDS; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A computer-implemented method for determining and displaying an access corridor to a target area in a brain of a patient, the method comprising:
a) generating a first image of the brain via positron emission tomography;
b) discriminating the target area relative to its surrounding via electronic image processing;
c) generating a second image of the brain via magnetic resonance imaging while acquiring at least one anatomical structure;
d) generating a third image of the brain via an imaging method displaying physiological processes for identifying at least one functional area of the brain that must not be injured in any circumstances;
e) determining an access corridor to the target area while omitting the at least one functional area of the brain; and
f) generating and displaying a fourth image of the brain, in which the target area, the at least one functional area of the brain, the at least one anatomical structure and the access corridor are displayed, wherein steps a) to d) are carried out, one after another in succession, in a single frame of reference without repositioning the patient.

2. The method as claimed in claim 1, wherein the third image in step d) is generated by way of at least one of dynamic positron emission tomography and functional magnetic resonance imaging.

3. The method as claimed in claim 1, wherein the at least one functional area of the brain is identified by at least one of diffusion weighted MRI and a BOLD image.

4. The method as claimed in claim 1, wherein the target area and the at least one functional area of the brain are visualized in different colors.

5. The method as claimed in claim 1, wherein the precision of the discrimination of the target area in the first image is improved in step b) by use of the second image.

6. The method as claimed in claim 1, wherein the frame of reference is provided by a stereotaxic frame.

7. A computer program product for, when executed on a control and evaluation system of an imaging arrangement, carrying out a method as claimed in claim 1.

8. A data storage medium including a computer program product, as claimed in claim 7.

9. An imaging arrangement for determining and displaying an access corridor to a target area in the brain of a patient, comprising:
a positron emission tomography imaging apparatus for generating a first image of the brain;
a magnetic resonance imaging apparatus for generating a second image of the brain while acquiring at least one anatomical structure;
an imaging apparatus imaging physiological processes for generating a third image of the brain and
a control and evaluation system for controlling the imaging arrangement and for determining an access corridor to the target area while omitting the at least one functional area of the brain and generating and displaying a fourth image of the brain, in which the target area, the at least one functional area of the brain, the at least one anatomical structure and the access corridor are displayed, wherein the first, second and third images are generated, one after another in succession, in a single frame of reference without repositioning the patient.

10. The method as claimed in claim 1, wherein at least one of the method steps is controlled by a control and evaluation system.

11. The method as claimed in claim 1, wherein steps a) to d) are carried out simultaneously.

12. The method as claimed in claim 10, wherein steps a) to d) are carried out simultaneously.

13. The method as claimed in claim 2, wherein the at least one functional area of the brain is identified by at least one of diffusion weighted MRI and a BOLD image.

14. The method as claimed in claim 2, wherein the target area and the at least one functional area of the brain are visualized in different colors.

15. A computer readable medium including program segments for,
when executed on a computer device, causing the computer device to implement the method of claim 1.

16. An imaging arrangement for determining and displaying an access corridor to a target area in the brain of a patient, comprising:
means for generating a first image of the brain via positron emission tomography;
means for discriminating the target area relative to its surrounding via electronic image processing;
means for generating a second image of the brain via magnetic resonance imaging while acquiring at least one anatomical structure;
means for generating a third image of the brain via an imaging method displaying physiological processes for identifying at least one functional area of the brain that must not be injured in any circumstances;
means for determining an access corridor to the target area while omitting the at least one functional area of the brain; and
means for generating and displaying a fourth image of the brain, in which the target area, the at least one functional area of the brain, the at least one anatomical structure and the access corridor are displayed, wherein the first, second and third images are generated, one after another in succession, in a single frame of reference without repositioning the patient.
